(19) United States
(12) Patent Application Publication
(10) Pub. No.: US 2025/0228991 A1
(43) Pub. Date: Jul. 17, 2025

(54) A wireless disinfecting apparatus using ozone

(71) Applicant: Carlos Jose Hinolan, Makati City (PH)

(72) Inventor: Carlos Jose Hinolan, Makati City (PH)

(21) Appl. No.: 18/037,842

(22) PCT Filed: Jan. 23, 2021
(86) PCT No.: PCT/PH2021/050002

§ 371 Date: May 19, 2023

(30) Foreign Application Priority Data
Nov. 19, 2020 (PH) 22020050631

(51) Int. Cl. A61L 2/20; A61L 9/015

(52) U.S. Cl. CPC A61L 2/202; A61L 9/015; A61L 2202/11; A61L 2202/14; A61L 2209/212

(57) ABSTRACT

An aspect of the invention is directed to a wireless ozone disinfecting apparatus that communicates wirelessly with a mobile computing device executing a software application and configured to determine pattern and operate the wireless ozone disinfecting apparatus remotely using an artificial intelligence data analytics system.

### TECHNICAL FIELD

The present invention relates generally to a wireless ozone disinfecting apparatus.

### BACKGROUND ART

The outbreak of COVID-19 worldwide in 2020 has increased the need for a reliable and efficient air disinfection method to decontaminate high-risk areas such as hospitals, schools, and other enclosed public places.

Ozone (O₃) or activated oxygen is known to kill microorganisms effectively. It is a naturally occurring gas found in the atmosphere, most commonly during lightning storms, at ocean beaches, and waterfalls. When exposed to electric high voltage, an oxygen molecule (O₂) is split onto two oxygen atoms (O₁). The oxygen atoms (O₁) then connects to oxygen molecules to form ozone (O₃). This ozone oxidation process is among one of the most efficient and most environmentally friendly way to kill microorganisms such as fungus, bacteria, and viruses.

Aside from being an effective biochemical disinfectant, ozone is also a powerful deodorizer. Ozone has a short half-life of about 30 minutes, which means that it reacts and disappears very fast, converting back to oxygen after disinfection. Advantages of using ozone disinfecting apparatus over a standard room disinfection using traditional disinfectants include no toxic chemical residues, low cost on material and consumables, and highly efficient.

Depending on the contamination level, the adequate ozone level for air disinfection of an unoccupied room is between 0.5 to 2.5 ppm.

Existing disinfecting apparatuses (US 20120230879 and U.S. Pat. No. 8,354,057) that use ozone are not portable and are not wirelessly connected to a remote device for activating the apparatus. Ozone gas elements are harmful to health; therefore, use of ozone disinfecting apparatus should not be in the proximity to any human being or animal.

In addition, the existing disinfecting apparatuses that use ozone do not utilize artificial intelligence in optimizing the use of the apparatuses and in turn efficiently disinfecting rooms. Further, incorrect amount of ozone may not kill harmful microorganisms.

### SUMMARY OF THE INVENTION

An aspect of the invention is directed to a wireless ozone disinfecting apparatus.

It is therefore an object of the invention to provide a wireless ozone disinfecting apparatus that can be accessed and activated remotely using a mobile computing device.

It is a further object of the invention to provide a wireless ozone disinfecting apparatus that uses artificial intelligence to ensure effective room disinfection.

### BRIEF DESCRIPTION OF THE DRAWINGS

The following drawings, which constitute a part of the description together with the implementations of the invention, are used to provide a further understanding of the invention, to explain the invention, but not to be taken in any manner as a limitation of the invention:

FIG. 1 is a perspective view of the wireless ozone disinfecting apparatus;

FIG. 2 is a side view of the wireless ozone disinfecting apparatus;

FIG. 3 is a top view of the wireless ozone disinfecting apparatus;

FIG. 4 is an exploded view of the wireless ozone disinfecting apparatus;

FIG. 5 is a diagram showing the connection of the parts of the wireless ozone disinfecting apparatus;

FIG. 6 is a diagram showing the network of wireless ozone disinfecting apparatus; and

FIG. 7 is an example interface of the software application for the wireless ozone disinfecting apparatus.

### DETAILED DESCRIPTION OF THE PREFERRED IMPLEMENTATIONS

All the ensuing disclosures and illustrations of the preferred implementations and/or embodiments of one or more aspects of the present invention, along with one or more components, features or elements thereof, are merely representative for the purpose of sufficiently describing the manner by which the present invention may be carried out into practice in various ways other than the ones outlined in the ensuing enabling description and specification of the present disclosure.

It is to be understood and appreciated, however, that the exemplary implementations used to describe how to make and use one or more aspects of the present invention may be embodied in many alternative forms and should not be construed as limiting the scope of the appended claims in any manner, absent express recitation of those features in the appended claims. All the exemplary drawings, diagrams, tables, formula, and illustrations accompanying the ensuing description should also not be construed as limiting the scope of the appended claims, as accompanied by this enabling description, in any manner.

Unless the context clearly and explicitly indicates otherwise, it is also to be understood that like reference numerals refer to like elements throughout the ensuing description of the figures and/or drawings of the present disclosure, that the linking term “and/or” includes any and all combinations of one or more of the associated listed items, that the singular terms “a”, “an” and “the” are intended to also include the plural forms, and that some varying terminologies of the same meaning and objective may be interchangeably used throughout the ensuing enabling disclosure of the present invention.

As may be used herein, unless otherwise specified, and consistent with one or more aspects and/or one or more implementations of the present invention, the term “include”, “consist” or “have” may refer to the corresponding component that can be inherent to exclude other components. All terms, including technical or scientific terms, have the same meaning as generally understood by a person skilled in the art to which the present invention belongs, unless otherwise defined. Commonly used terms, such as herein predefined terms, should be interpreted as being consistent with the meaning of the context of the related art, and are not to be interpreted as ideal or excessively formal meanings unless explicitly defined in the present disclosure.

As may be used herein, unless otherwise specified, and consistent with one or more aspects and/or one or more implementations of the present invention, the term “implementation” may generally refer to selection, observation, collection, preservation, maintenance, organizing, integration, visualization, mapping, categorizing, inspecting, analyzing, interpreting, classifying, sorting, filtering, prioritizing, controlling, extracting, sharing, navigating, retrieving, contextualizing, recording and/or archiving of any number and type of electronic items or electronic media items.

As may be used herein, unless otherwise specified, and consistent with one or more aspects and/or one or more implementations of the present invention, the terms “connected to,” or “in communication with,” may include direct connection/communication, indirect connection/communication and/or inferred connection/communication between devices/apparatuses/computers. The direct connection/communication may be provided through one or more hardware, software, firmware, electronic and/or electrical links between devices/apparatuses/systems. The indirect connection/communication may be provided through an intervening member such as a component, an element, a circuit, a module, a device, a node device, and an apparatus between or among devices/apparatuses/systems. The inferred connection/communication may be characterized by one device/apparatus/system being connected to or in operative communication with another device/apparatus/system by inference, and may accordingly include direct and indirect connections/communications.

The present invention relates to a wireless ozone disinfecting apparatus 100 comprising a first and second one air pressuring means 104, 106 for pressuring air and releasing ozone, a fan 108 for creating air flow to circulate ozone and disposed relative to the air pressuring means, an ozone generator 110 for producing ozone in operative communication with the fan 108 and configured to produce ozone, a hardware processor 116 disposed in operative communication with the ozone generator 110 and fan 108 and configured to selectively activate the ozone generator 110 and fan 108 and a power source 112 in electrical communication with the hardware processor 116 and configured to provide electrical power to the wireless ozone disinfecting apparatus 100, wherein the hardware processor 116 communicates wirelessly with a mobile computing device 118 executing a software application and configured to send and receive data to and from the hardware processor 116 via the communications module 116c.

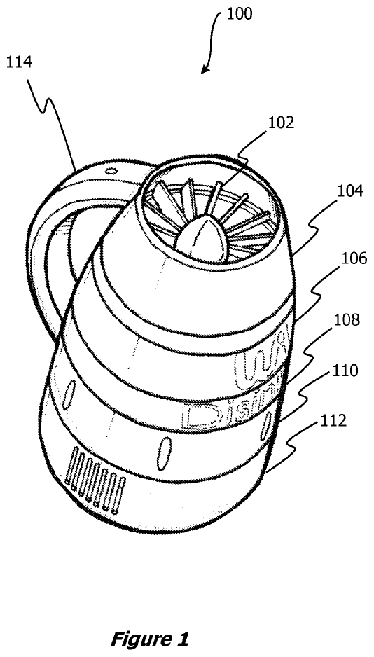

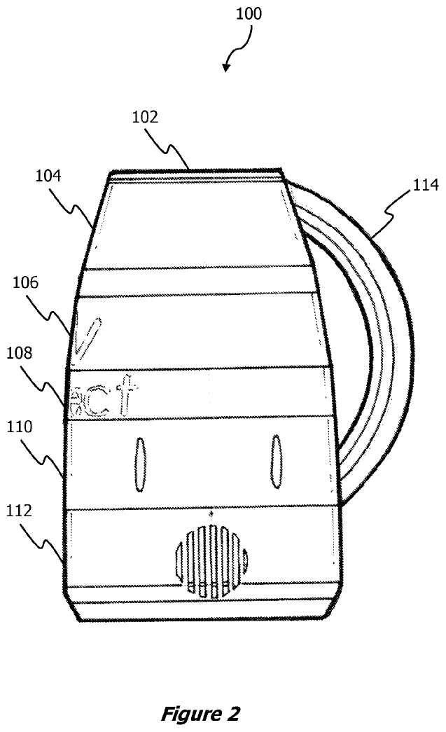

FIGS. 1 and 2 shows a preferred implementation of a wireless ozone disinfecting apparatus 100 with a power source 112 at the bottom portion of the apparatus. The power source 112 may be disposed at the bottom in order for batteries and/or for power cord to be secured in place. The power source may also be in the form of USB charger to set up with a portable powerbank or laptop.

The power source 112 includes a hardware processor 116 (not shown) that activates the ozone generator 110 located above the power source 112. The fan 108 is preferably located above the ozone generator 110 for creating an upward airflow from the ozone generator 110 through the first air pressuring means 106 that is a funnel-shaped cylinder above the fan 108. The funnel-shaped outlet 104 at the top portion of the wireless disinfecting apparatus 100 has fins 102 on top to prevent debris from coming inside the wireless ozone disinfecting apparatus 100. The wireless ozone disinfecting apparatus 100 also has a handle 114 for carrying means.

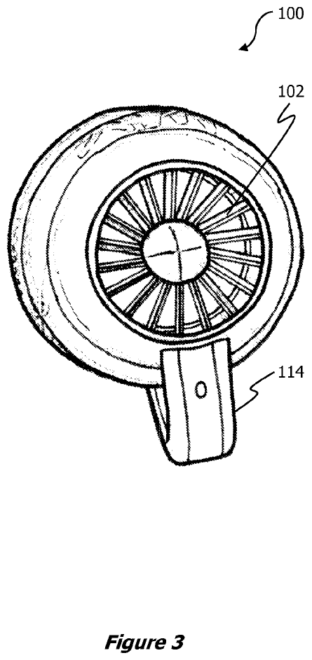

In FIG. 3, there is shown the top view of the wireless ozone disinfecting apparatus 100 emphasizing the fins 102 to catch any debris that may fall into the apparatus. Instead of fins, the top portion of the wireless ozone disinfecting apparatus 100 may be in the form of mesh or any type of barrier against unwanted materials getting inside the wireless ozone disinfecting apparatus 100.

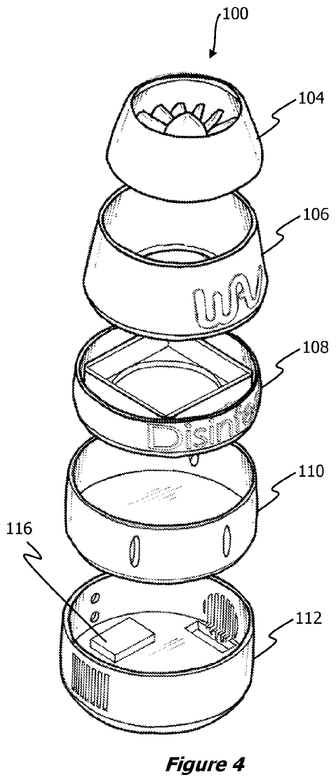

FIG. 4 shows an exploded view of the wireless ozone disinfecting apparatus 100 to highlight the layers by which the parts are connected to each other. The bottom part which is the power source 112 may contain the hardware processor 116. The power source 112 may be in electrical communication with the hardware processor 116 and may be disposed at the lower portion of the wireless ozone disinfecting apparatus 100 in order to maintain the stability.

The hardware processor 116 may be disposed in operative communication with the ozone generator 110 and the fan 108. The hardware processor 116 may be configured to selectively activate the ozone generator 110. The ozone generator 110 may be configured to trigger the surrounding oxygen to split into two and then form into ozone. The hardware processor 116 may also be configured to selectively activate the fan 108 that creates airflow. The ozone generated by the ozone generator 110 may be drawn by the fan 108 towards the funnel-shaped outlet 104 and into the room being disinfected. The ozone generated by the ozone generator 110 may flow through the funnel-shaped cylinder 106, utilizing the principle of Venturi effect, to efficiently eject the ozone to the room, or any given space, being disinfected.

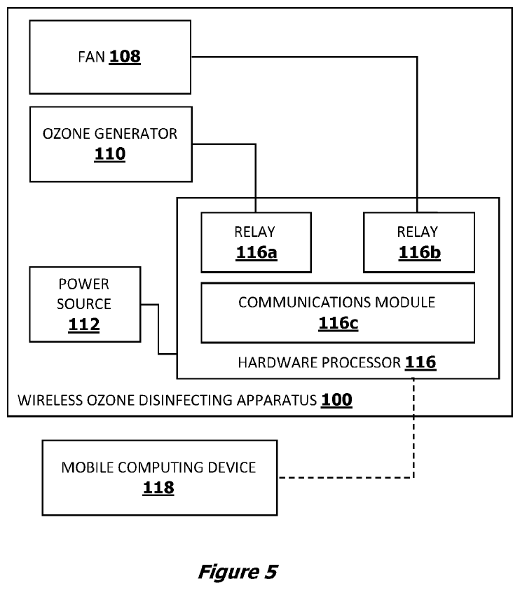

In FIG. 5, there is shown an example and a diagram of the connection within the wireless ozone disinfecting apparatus 100. The wireless ozone disinfecting apparatus 100 may contain the basic parts such as fan 108, ozone generator 110, power source 112, and hardware processor 116. The power source 112 may be in operable communication with the hardware processor 116 that is in operative communication with the ozone generator 110 and the fan 108. The power source 112 may be in the form of alternating current (AC) or direct current (DC).

The hardware processor may be in electrical communication with the ozone generator 110 and the fan 108 through an ozone generator relay 116a, and a fan relay 116b. The hardware processor 116 may be provided with a communications module 116c to enable the wireless ozone disinfecting apparatus 100 to communicate with the mobile computing device 118. The hardware processor 116 may be configured to store software codes needed to operate the wireless ozone disinfecting apparatus 100 and communicate with the mobile computing device 118. The mobile computing device 118 may be configured to send and receive data to and from the wireless ozone disinfecting apparatus 100 via the communications module 116c of the hardware processor 116. Consistent with the present invention, the hardware processor is configured to store software codes and process log data indicating the operations of the wireless ozone disinfecting apparatus.

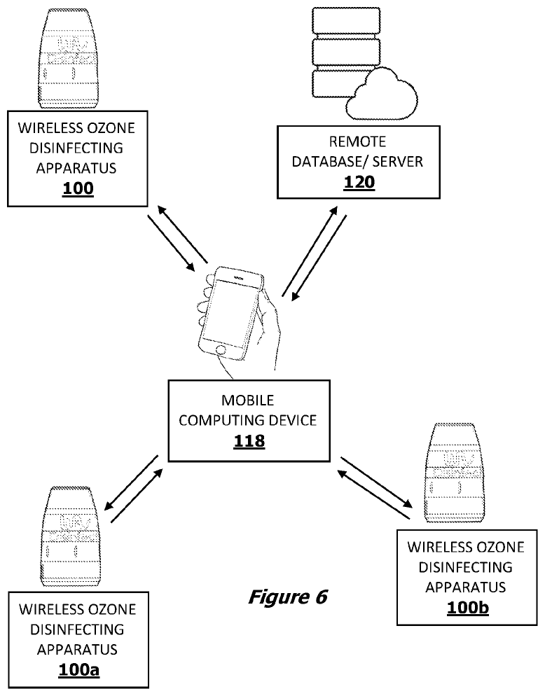

As shown in FIG. 6 and as an example implementation, the wireless ozone disinfecting apparatus 100 may be in communication with a mobile computing device 118. The mobile computing device 118 may also be in communication with a second wireless ozone disinfecting apparatus 100a and a third wireless ozone disinfecting apparatus 100b to create a network of wireless disinfecting apparatuses through a dedicated software application installed in the mobile computing device 118. Consistent with the present invention, the hardware processor is configured to communicate with other hardware processor of at least one wireless ozone disinfecting apparatus to create a network of wireless ozone disinfecting apparatuses.

The mobile computing device 118 having a dedicated software application may be configured to access the log data stored on the hardware processor 116 of the wireless ozone disinfecting apparatus 100. Consistent with the present invention, the mobile computing device executing a software application is configured to access the log data. The mobile computing device 118 may utilize artificial intelligence in generating new information based on the accessed log data and may transmit the new information to a remote database or server 120. The new information generated by in the form of pattern and/or instruction that is transmittable to the wireless ozone disinfecting apparatus 100 and the remote database server 120. The remote database server 120 may store information and may send instructions to the wireless ozone disinfecting apparatus 100 through the mobile computing device 118 via the communications network. Consistent with the present invention, the mobile computing device 118 executing a software application is configured to determine pattern and operate the wireless ozone disinfecting apparatus 100 remotely using an artificial intelligence data analytics system.

While the present disclosure includes several variations described in detail, various modifications and improvements thereon will become readily apparent to those skilled in the art. Accordingly, the spirit and scope of the present invention is not to be limited by the foregoing examples and preferred implementations, but is to be understood in the broadest sense allowable by relevant laws and rules.

Further, the scope of the present invention is shown by the following or accompanying claims rather than the above description which is illustrative in nature, and all changes or modifications derived from the meaning and scope of the claims and their equivalents should be construed as being included in the scope of the present invention.

1. A wireless ozone disinfecting apparatus comprising:
a first and second air pressuring means for pressuring air and releasing ozone;
a fan for creating air flow to circulate ozone and disposed relative to the air pressuring means;
an ozone generator for producing ozone in operative communication with the fan and configured to produce ozone;
a hardware processor disposed in operative communication with the ozone generator and fan and configured to selectively activate the ozone generator and fan; and
a power source in electrical communication with the hardware processor and configured to provide electrical power to the wireless ozone disinfecting apparatus;
wherein the hardware processor communicates wirelessly with a mobile computing device executing a software application and configured to send and receive data to and from the hardware processor via the communications module.

2. The wireless ozone disinfecting apparatus according to claim 1, wherein the hardware processor is configured to store software codes to store log data indicating the operations of the wireless ozone disinfecting apparatus;

3. The wireless ozone disinfecting apparatus according to claim 2, wherein the mobile computing device executing a software application is configured to access the log data;

4. The wireless ozone disinfecting apparatus according to claim 3, wherein the mobile computing device executing a software application is configured to determine pattern and operate the wireless ozone disinfecting apparatus remotely using an artificial intelligence data analytics system.

5. The wireless ozone disinfecting apparatus according to claim 1, wherein the hardware processor is configured to communicate with other hardware processor of at least one wireless ozone disinfecting apparatus to create a network of wireless ozone disinfecting apparatuses.
